(19) United States
(12) Patent Application Publication
(10) Pub. No.: US 2026/0102570 A1
(43) Pub. Date: Apr. 16, 2026

(54) NEBULIZER CONTROLLER WITH POWER-ON FREQUENCY SWEEPS FUNCTION

(71) Applicant: VADI Medical Technology Co., Ltd., Taoyuan City (TW)

(72) Inventor: Wei-Shin HUANG, Taoyan City (TW)

(21) Appl. No.: 19/019,794

(22) Filed: Jan. 14, 2025

(30) Foreign Application Priority Data
Oct. 14, 2024 (TW) 113211053

(51) Int. Cl. A61M 11/00

(52) U.S. Cl. CPC A61M 11/005; A61M 2202/0007; A61M 2202/04; A61M 2205/3327; A61M 2205/50

(57) ABSTRACT

A nebulizer controller with power-on frequency sweeps function comprises a USB plug, a controller, and a nebulizer head. The input end of the controller is electrically connected with the USB plug to receive electric power, while the output end is electrically connected with the nebulizer head to transmit electric power and signal. The controller comprises a current sensing circuit, a micro-controller, and a drive circuit. The micro-controller is configured with a plurality of drive frequencies to be sequentially provided to the drive circuit. The current sensing circuit measures the value of the current consumed by each drive frequency and feeds it back to the micro-controller for recording. The micro-controller sweeps and locks the drive frequency corresponding to the maximal current value as the optimal oscillating frequency for aerosolization by the nebulizer head.

### CROSS-REFERENCE TO RELATED APPLICATION

The present application claims priority under 35 USC 119 to Taiwan utility patent application No. 113211053, filed Oct., 14, 2024, which is hereby incorporated by reference in its entirety.

### BACKGROUND

### Technical Field

The present invention relates to a nebulizer, and more particularly to a controller with power-on frequency sweeps fuction to lock the optimal oscillating frequency for aerosolization whenever the nebulizer is turned on.

### Description of Related Arts

A nebulizer operates vibrations to aerosolize medicinal liquid into aerosol, which is inhaled into the respiratory passage to cure bronchiectasis, diminish inflammation, or reduce phlegm. As shown in FIG. 1, U.S. Pat. No. 10,029,053 discloses a nebulizer 1 comprising a controller 2 connected to the nebulizer head 3. The nebulizer 1 comprises a USB plug 4 for connection, cables between the USB plug 4 and the controller 2, and other cables between the controller 2 and the nebulizer head 3.

The controller 2 comprises a voltage boost circuit 10, a controller 11, and a drive circuit 12. From the range of 4.75V to 5.25V DC, the voltage boost circuit 10 inputs a voltage of 12V DC required for the drive circuit 12 to generate drive frequencies ranging from 120 to 150 kHz to drive the nebulizer head 3 to generate sufficient amplitude of vibration to aerosolize the medicinal liquid. However, upon each power-on of the nebulizer and initial power supply by the USB plug 4, the controller 2 does not sweep frequencies and obtain the optimal drive frequency currently required by the vibrating plate of the nebulizer head, whereas inappropriate drive frequencies may cause temperature rise due to excessive vibration of the nebulizer head. Once the temperature rise exceeds 10° C., the quality of the medicinal liquid will be dramatically affected.

### BRIEF SUMMARY OF THE INVENTION

The Inventor is a professional person of medical devices and has rich experience in this industry. In view of the problem of temperature rise caused by excessive vibration of the vibrating plate of the nebulizer head due to inappropriate drive frequencies of the controller and the consequent influence to the quality of the medicinal liquid. An improved controller with frequency sweeps function whenever the nebulizer is power-on to supply electric power to the USB plug and lock the optimal frequency for the nebulizer head to aerosolize the medicinal liquid to effectively solve the above-mentioned problem.

The present invention provides a controller with power-on frequency sweeps, which comprises a USB plug, a controller, and a nebulizer head, the input end of the controller being electrically connected with the USB plug to receive electric power, while the output end is electrically connected with the nebulizer head to transmit electric power and signal. The controller comprises a current sensing circuit, a micro-controller, and a drive circuit. The micro-controller is configured with a plurality of drive frequencies to be sequentially provided to the drive circuit. The current sensing circuit measures the value of current consumed by each drive frequency and feeds it back to the micro-controller for recording. The micro-controller sweeps and locks the drive frequency corresponding to the maximal current value as the optimal oscillating frequency for aerosolization.

Other objects, features, and advantages of the present application will become apparent from the following detailed description, the accompanying drawings, and the appended claims.

### BRIEF DESCRIPTION OF THE DRAWINGS

FIG. 1 is block diagram of the prior art;

FIG. 2 is block diagram of the present invention.

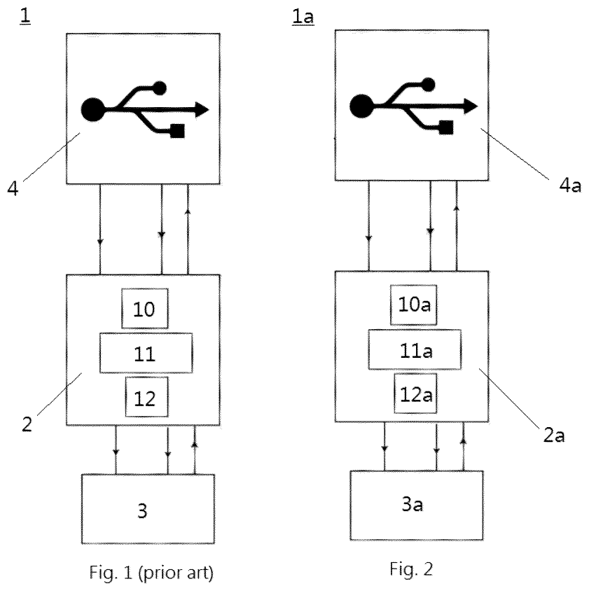

### DETAILED DESCRIPTION OF THE INVENTION

Referring to FIG. 2, the present invention comprises a USB plug 4a, a controller 2a, and a nebulizer head 3a. The input end of the controller 2a is electrically connected with the USB plug 4a to receive electric power, while the output end is electrically connected with the nebulizer head 3a to transmit electric power and signal.

Upon power-on, the USB plug 4a transmits electric power to the controller 2a and the nebulizer head 3a. The controller 2a comprises a current sensing circuit 10a, a micro-controller 11a, and a drive circuit 12a. The micro-controller 11a is configured with a plurality of drive frequencies to be sequentially provided to the drive circuit 12a. The current sensing circuit 10a measures the value of current consumed by each drive frequency and feeds it back to the micro-controller 11a for recording. The micro-controller sweeps and locks the drive frequency corresponding to the maximal current value as the optimal oscillating frequency for aerosolization by the nebulizer head.

In summary, the core value of the present invention lies in the provision of a nebulizer, in which the controller sweeps the frequencies whenever the nebulizer is power-on, and locks the optimal oscillating frequency for aerosolization by the nebulizer head, so as to avoid temperature rise due to excessive vibration of the nebulizer head resulting from inappropriate drive frequencies.

Although the present patent application has been described in detail for the purpose of illustration, it is to be understood that such detail is solely for that purpose and that the present patent application is not limited to the disclosed embodiment, but, on the contrary, is intended to cover modifications and equivalent arrangements that are within the spirit and scope of the appended claims.

What is claimed is:

1. A nebulizer controller with power-on frequency sweeps fuction, comprising a USB plug, a controller, and a nebulizer head, the input end of the controller being electrically connected with the USB plug to receive electric power, while the output end being electrically connected with the nebulizer head to transmit electric power and signal, wherein:
the controller comprises a current sensing circuit, a micro-controller, and a drive circuit, the micro-controller is configured with a plurality of drive frequencies to be sequentially provided to the drive circuit, the current sensing circuit measures the value of current consumed by each drive frequency and feeds it back to the micro-controller for recording, the micro-controller sweeps and locks the drive frequency corresponding to the maximal current value as the drive frequency for aerosolization by the nebulizer head.
